(19) United States
(12) Patent Application Publication
(10) Pub. No.: US 2011/0015265 A1
(43) Pub. Date: Jan. 20, 2011

(54) METHOD FOR CONTROLLING DISEASES ON TURFGRASS

(75) Inventor: Masanao TAKAISHI, Toyonaka-shi (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHEMICAL COMPANY, LIMITED, Tokyo (JP)

(21) Appl. No.: 12/835,121

(22) Filed: Jul. 13, 2010

(30) Foreign Application Priority Data
Jul. 14, 2009 (JP) 2009-165366

(51) Int. Cl. A01N 37/10; A01N 37/18; C07C 69/734; C07C 235/34; A01P 1/00

(52) U.S. Cl. 514/532; 514/622; 560/60; 564/182

(57) ABSTRACT

The present invention provides a method for controlling diseases on turfgrass which comprises applying to turfgrass that has been cut back an effective amount of a compound of formula (1).

### TECHNICAL FIELD

The present invention relates to a method for controlling diseases on turfgrass.

### BACKGROUND ART

It is known from WO 95/27693 and WO 96/07633 that certain a-Substituted phenylacetic acid compounds can be used as active ingredients of fungicides. While these compounds are known to be effective in controlling diseases on turfgrass, there remains a need for novel methods to enhance efficacy of turfgrass disease control.

### disclosure of invention

An object of the present invention is to provide a method for controlling diseases on turfgrass.

The present invention provides a method for controlling diseases on turfgrass, having an improved conₜrol efficacy for turfgrass diseases by applying a compound of the following formula (1) to shaven turfgrass.

Specifically, the present invention provides:

[1] a method for controlling diseases on turfgrass which comprises applying to turfgrass that has been cut back an effective amount of a compound of formula (1):

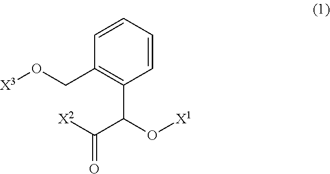

wherein X′ represents a methyl group, a difluoromethyl group or an ethyl group; X² represents a methoxy group or a methylamino group; and X³ represents a phenyl group, a 2-methylphenyl group or a 2,5-dimethylphenyl group (hereinafter, referred to as the present compound in some cases);

[2] the method according to [1], wherein the turfgrass is not more than 35 millimeters but not less than 5 millimeters in height;

[3] the method according to [1], wherein the turfgrass is not more than 12 millimeters but not less than 5 millimeters in height; and

[4] use of the compound of formula (1) of [1] for applying to turfgrass that has been cut back to control diseases on the turfgrass.

Diseases on turfgrass can be controlled according to the method of the present invention.

### MODES FOR CARRYING OUT THE INVENTION

Examples of the present compound for use in the present invention include the following compounds:

a compound in which X¹ is a methyl group, a difluoromethyl group or an ethyl group in formula (1);

a compound in which X¹ is a methyl group in formula (1);

a compound in which X² is a methoxy group or a methylamino group in formula (1);

a compound in which X¹ is a methyl group and X² is a methoxy group in formula (1);

a compound in which X¹ is a methyl group and X² is methylamino group in formula (1);

a compound in which X³ is a phenyl group, a 2-methylphenyl group or a 2,5-dimethylphenyl group in formula (1);

a compound in which X³ is a phenyl group or a 2,5-dimethylphenyl group in formula (1);

a compound in which X′ is a methyl group, X² is a methoxy group, and X³ is a 2,5-dimethylphenyl group in formula (1);

a compound in which X′ is a methyl group, X² is a methylamino group, and X³ is a phenyl group in formula (1); and

a compound in which X¹ is a methyl group, X² is a methylamino group, and X³ is a 2,5-dimethylphenyl group in formula (1).

Specific examples of the present compound are shown below.

In the compound of formula (1), X¹, X² and X³ are one of the combinations of members shown in Table 1.

TABLE 1

| The present compound | X¹ | X² | X³ |
|---|---|---|---|
| The present compound (1) | CH₃ | OCH₃ | Ph |
| The present compound (2) | CH₃ | OCH₃ | 2-CH₃Ph |
| The present compound (3) | CH₃ | OCH₃ | 2,5-(CH₃)₂Ph |
| The present compound (4) | CH₃ | NHCH₃ | Ph |
| The present compound (5) | CH₃ | NHCH₃ | 2-CH₃Ph |
| The present compound (6) | CH₃ | NHCH₃ | 2,5-(CH₃)₂Ph |
| The present compound (7) | CHF₂ | OCH₃ | Ph |
| The present compound (8) | CHF₂ | OCH₃ | 2-CH₃Ph |
| The present compound (9) | CHF₂ | OCH₃ | 2,5-(CH₃)₂Ph |
| The present compound (10) | CHF₂ | NHCH₃ | Ph |
| The present compound (11) | CHF₂ | NHCH₃ | 2-CH₃Ph |
| The present compound (12) | CHF₂ | NHCH₃ | 2,5-(CH₃)₂Ph |
| The present compound (13) | C₂H₅ | OCH₃ | Ph |
| The present compound (14) | C₂H₅ | OCH₃ | 2-CH₃Ph |
| The present compound (15) | C₂H₅ | OCH₃ | 2,5-(CH₃)₂Ph |
| The present compound (16) | C₂H₅ | NHCH₃ | Ph |
| The present compound (17) | C₂H₅ | NHCH₃ | 2-CH₃Ph |
| The present compound (18) | C₂H₅ | NHCH₃ | 2,5-(CH₃)₂Ph |

The present compound may have isomers such as tautomers and stereoisomers including optical isomers based on an asymmetric carbon atoms, and any isomer can be contained and used singly or in a mixture of any isomer ratio in the present invention.

The present compound may be in a form of a solvate (for example, hydrate) and it can be used in a form of a solvate in the present invention.

The present compound may be in a form of a crystal form and/or an amorphous form and it can be used in any form in the present invention.

The present compounds are compounds described in WO95/27693 pamphlet. These compounds can be synthesized, for example, by a method described in the pamphlet.

The method of the present invention is characterized by applying the present compound to turfgrass that has been cut back. As used in the present specification, “turfgrass that has been cut back” refers to turfgrass that has been cut back to be prescribed range in height. The maximum height of the turfgrass is preferably 35 millimeters and more preferably 12 millimeters. The minimum height of the turfgrass may be in a level that would not cause growth retardation of the turfgrass, and is specifically about 5 millimeters.

Cutting of turfgrass may be done by any method conventionally used in lawn care or turf management without any particular limitation. For golf course maintenance, mowers are used, for example, greens mowers are used for cutting of greens or teeing ground, approach mowers or riding approach mowers are used for cutting of approach, reel mowers or gang fairway mowers are used for cutting of fairway, and rotary mowers are used for cutting rough.

In the case of applying the present compound in the method of the present invention, diseases on turfgrass can be controlled by applying an effective amount of the present compound to a place where the pathogens of turfgrass diseases inhabit or may inhabit, specifically by applying the present compound onto turf. As used herein “turf” refers to a group of turfgrass, which covers a surface area of ground and is subject to regular maintenance. The application may be done by any method conventionally used for treatment of turfgrass with fungicides without any particular limitation, and examples of the treatment method include spraying by using horticultural watering can, hand-compression sprayer or power sprayer. The present compound may be applied by mixing it into an irrigation liquid, for example, by injecting to irrigation facilities such as irrigation tube, irrigation pipe and sprinkler.

Examples of turfgrass to which the method of the present invention may be applied include turfgrasses planted in golf courses, parks or private gardens. Examples of ₜhe turfgrass also include seedling of turfgrass. Examples of the place for treating seedling include seedling raising box, seedling raising tray and seedbed, and examples of the treating period include raising period, before settled planting, at the time of settled planting, and growing period after settled planting.

Examples of turfgrass to which the method of the present invention may be applied include the following species of turfgrasses.

Examples of warm season turfgrasses include: Zoysiagrasses (Zoysia spp.) such as Zoysia japonica, Zoysia matrella and Zoysia tenuifolia; Bermudagrasses (Cynodon spp.); Centipedegrass (Eremochloa ophiuroides); and St. Augustinegrass (Stenotaphrum secundatum).

Examples of cool season turfgrasses include: Bentgrasses (Agrostis spp.) such as Colonial Bentgrass (Agrostis tenuis), Creeping Bentgrass (Agrostis palustris), Velvet Bentgrass (Agrostis canina) and Redtop (Agrostis alba); Ryegrasses (Lolium spp.) such as Italian Ryegrass (Lolium multiflorum) and Perennial Ryegrass (Lolium perenne); Bluegrasses (Poa spp.) such as Kentucky Bluegrass (Poa pratensis) and Rough Bluegrass (Poa tribialis); and Fescues (Festuca spp.) such as Red Fescue (Festuca rubra) and Tall Fescue (Festuca arundinacea).

Examples of turfgrass diseases which can be controlled by the method of the present invention include the following phytopathogenic diseases on turfgrasses: Curvularia leaf blight (Curvularia geniculata), Drechslera leaf spot (Drechslera gigantea), Pink snow mold (Microdochium nivale), Rust (Puccinia zoysiar), Southern blight (Sclerotium rolfsii), Large patch (Rhizoctonia solani), Fairy rings (Lycoperdon perlatum, Lepista subnuda, Marasmius oreades, Agaricus campestris), Pythium red blight (Pythium aphanidermatum), Pythium snow blight (Pythium iwayamai, Pythium paddicum), Crown rust (Puccinia coronata), Brown ring patch (Rhizoctonia oryzae), Brown stripe (Sclerotinia graminis), Take-all (Gaumannomyces graminis), Dollar spot (Sclerotinia homoeocarpa), Anthracnose (Colletotricum graminicola), Brown patch (Rhizoctonia solani), Pythium blight (Pythium graminicola, Pythium vanterpoolii, Pythium aristosporum), Typhula snow blight (Typhla incarnata), Typhula snow blight (Typhla ishikariensis) and Sclerotinia Snow blight (Sclerotinia borealis).

Particularly, the method of the present invention is expected to be highly effective at controlling such phytopathogenic diseases as Pink snow mold (Microdochium nivale), Large patch (Rhizoctonia solani), Pink snow mold (Microdochium nivale) and Dollar spot (Sclerotinia homoeocarpa).

When the present compound is used in the method of the present invention, the present compound may be used singly. Typically, the present compound is used in the form of an oil agent, an emulsion, a flowable agent, a wettable powder, a granulated wettable powder, a powder agent, a granule agent or the like, which can be formulated by mixing the present compound with an inert carrier and adding to the mixture a surfactant and other adjuvants as needed. In these formulations, the amount of the present compound is typically in the range of 0.1% to 99% by weight, preferably 0.2% to 90% by weight.

Examples of the solid carrier used in formulation include fine powders or granules such as minerals such as kaolin clay, attapulgite clay, bentonite, montmorillonite, acid white clay, pyrophyllite, talc, diatomaceous earth and calcite; natural organic materials such as corn rachis powder and walnut husk powder; synthetic organic materials such as urea; salts such as calcium carbonate and ammonium sulfate; synthetic inorganic materials such as synthetic hydrated silicon oxide; and as a liquid carrier, aromatic hydrocarbons such as xylene, alkylbenzene and methylnaphthalene; alcohols such as 2-propanol, ethyleneglycol, propylene glycol, and ethylene glycol monoethyl ether; ketones such as acetone, cyclohexanone and isophorone; vegetable oil such as soybean oil and cotton seed oil; petroleum aliphatic hydrocarbons, esters, dimethylsulfoxide, acetonitrile and water.

Examples of the surfactant include anionic surfactants such as alkyl sulfate ester salts, alkylaryl sulfonate salts, dialkyl sulfosuccinate salts, polyoxyethylene alkylaryl ether phosphate ester salts, lignosulfonate salts and naphthalene sulfonate formaldehyde polycondensates; and nonionic surfactants such as polyoxyethylene alkyl aryl ethers, polyoxyethylene alkylpolyoxypropylene block copolymers and sorbitan fatty acid esters and cationic surfactants such as alkyltrimethylammonium salts.

Examples of the other formulation auxiliary agents include water-soluble polymers such as polyvinyl alcohol and polyvinylpyrrolidone, polysaccharides such as Arabic gum, alginic acid and the salt thereof, CMC (carboxymethyl-cellulose), Xanthan gum, inorganic materials such as aluminum magnesium silicate and alumina sol, preservatives, coloring agents and stabilization agents such as PAP (acid phosphate isopropyl) and BHT.

While the amounts of the present compound used in the method of the present invention may be changed depending on the kind of the turfgrass to be treated, the kind and the occurring frequency of the turfgrass diseases to be controlled, formulation form, treatment period, climatic condition and so on, the amount of the present compound per 10,000 m² is typically 1 g to 5,000 g and preferably 2 g to 1,000 g.

The emulsion, wettable powder and flowable agent are typically diluted with water, and then sprinkled for the treatment. In these case, the concentration of the present compound is typically in the range of 0.0001% to 3% by weight and preferably 0.0005% to 1% by weight. The powder agent and granule agent are typically used for the treatment without being diluted.

### Examples

While the present invention will be more specifically described below, the present invention is not limited to the following examples. In the following examples, the part represents part by weight unless otherwise noted in particular.

The compound (6) is the present compound (6) shown in Table 1 and a racemic form of the compound in which X′ is a methyl group, X² is a methylamino group and X³ is a 2,5-dimethylphenyl group in formula (1).

Formulation example 1

Fully mixed are 2.5 parts of the compound (6), 14 parts of polyoxyethylene styrylphenyl ether, 6 parts of calcium dodecyl benzene sulfonate and 83.5 parts of xylene, so as to obtain an emulsion.

Formulation example 2

Five (5) parts of the compound (6), 35 parts of a mixture of white carbon and a polyoxyethylene alkyl ether sulfate ammonium salt (weight ratio 1:1) and 60 parts of water are mixed, and the mixture is subjected to fine grinding according to a wet grinding method, so as to obtain a flowable formulation.

Formulation example 3

Five (5) parts of the compound (6), 1.5 parts of sorbitan trioleate and 38.5 parts of an aqueous solution containing 2 parts of polyvinyl alcohol are mixed, and the mixture is subjected to fine grinding according to a wet grinding method. Thereafter, 45 parts of an aqueous solution containing 0.05 part of Xanthan gum and 0.1 part of aluminum magnesium silicate is added to the resultant mixture, and 10 parts of propylene glycol is further added thereto. The obtained mixture is blended by stirring, so as to obtain a flowable formulation.

Formulation example 4

Forty (40) parts of the compound (6), 5 parts of propylene glycol (manufactured by Nacalai Tesque), 5 parts of SoprophorFLK (manufactured by Rhodia Nikka), 0.2 parts of an anti-form C emulsion (manufactured by Dow Corning), 0.3 parts of proxel GXL (manufactured by Arch Chemicals) and 49.5 parts of ion-exchange water are mixed so as to obtain a bulk slurry. Into 100 parts of the slurry, 150 parts of glass beads (diameter =1 mm) are put and the slurry is ground for 2 hours while being cooled with a cooling water. After ground, the resultant is filtered to remove the glass beads and a flowable formulations is obtained.

Formulation example 5 Three (3) parts of calcium lignin sulfonate, 12.5 parts of the compound (6), 2 parts of sodium lauryl sulfate and 84.5 parts of synthetic hydrated silicon oxide are fully ground and mixed so as to obtain wettable powders.

Formulation example 6

Five (5) parts of the compound (6), 1 part of synthetic hydrated silicon oxide, 2 parts of calcium lignin sulfonate, 30 parts of bentonite and 62 parts of kaolin clay are fully ground and mixed, and the resultant mixture is added with water and fully kneaded, and then subjected to granulation and drying so as to obtain a granule formulation.

Formulation example 7

Five (5) parts of the compound (6), 85 parts of kaolin clay and 10 parts of talc are fully ground and mixed so as to obtain a powder formulation.

Test example 1

A plastic pot was filled with sandy soil. Turfgrasses (bentgrass penncross) were disseminated and allowed to grow in a green house for 20 days, and then cut back to a prescribed height. A flowable formulation of the compound (6) was prepared according to the formulation example 2, and the obtained flowable formulation was diluted with water to prepare a diluted solution containing prescribed concentration of the compound (6). The diluted solution was subjected to foliage application by spraying it onto the turfgrasses in the ratio of 1,000 L per 10,000 m². After the turfgrasses were air-dried, Sclerotinia homoeocarpa, pathogen of dollar spot of bentgrass, which had been cultured on a bran medium was inoculated to the turfgrasses in the ratio of about 30 mg per pot. Then, the pot of turfgrasses were placed at 19° C. to 23° C. under high humidity for 6 days, and thereafter diseased area ratio was checked.

As a comparison, a formulation of Insignia (registered trademark)(manufactured by BASF Corporation, the active ingredient is pyraclostrobin) was diluted with water in prescribed concentration to prepare a diluted solution and it was subjected to a test similar to the above.

The control value was calculated by Equation 1. The results are shown in Table 2.

“Equation 1”

Control value =100 x (A-B)/A A: Diseased area ratio of plants treated with none of the test compounds B: Diseased area ratio of plants treated with a test compound

TABLE 2

| | | | | Relative |
|---|---|---|---|---|
| | Concentration | Height of | Control | control |
| Test compound | (g/ha) | turfgrass | value | value* |
| The compound (6) | 150 | 5 to 12 mm | 100 | 114 |
| The compound (6) | 37.5 | 5 to 12 mm | 100 | 114 |
| The compound (6) | 9.4 | 5 to 12 mm | 95 | 179 |
| The compound (6) | 2.3 | 5 to 12 mm | 69 | 238 |
| The compound (6) | 150 | 36 mm or more | 88 | 100 |
| The compound (6) | 37.5 | 36 mm or more | 88 | 100 |
| The compound (6) | 9.4 | 36 mm or more | 53 | 100 |
| The compound (6) | 2.3 | 36 mm or more | 29 | 100 |
| Insignia | 150 | 5 to 12 mm | 85 | 104 |
| Insignia | 37.5 | 5 to 12 mm | 69 | 97 |
| Insignia | 150 | 36 mm or more | 82 | 100 |
| Insignia | 37.5 | 36 mm or more | 71 | 100 |

*Relative control value is shown by setting as 100 a control value of plants with a height of 36 mm or more and treated with the same concentration of the same compound.

### INDUSTRIAL APPLICABILITY

According to the method of the present invention, turfgrass can be protected against phytopathogenic diseases.

What is claimed is:

1. A method for controlling diseases on turfgrass which comprises applying to turfgrass that has been cut back an effective amount of a compound of formula (1): 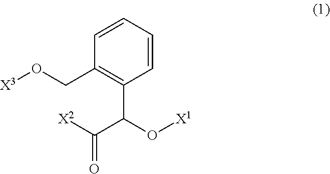
wherein X¹ represents a methyl group, a difluoromethyl group or an ethyl group; X² represents a methoxy group or a methylamino group; and X³ represents a phenyl group, a 2-methylphenyl group or a 2,5-dimethylphenyl group.

2. The method according to claim 1, wherein the turfgrass is not more than 35 millimeters but not less than 5 millimeters in height.

3. The method according to claim 1, wherein the turfgrass is not more than 12 millimeters but not less than 5 millimeters in height.

4. Use of the compound of formula (1) of claim 1 for applying to turfgrass that has been cut back to control diseases on the turfgrass.
